(19) United States
(12) Patent Application Publication
(10) Pub. No.: US 2026/0217109 A1
(43) Pub. Date: Jul. 30, 2026

(54) ALCOHOL INTAKE DETERMINING DEVICE AND ALCOHOL INTAKE DETERMINING METHOD

(71) Applicant: Mitsubishi Electric Mobility Corporation, Tokyo (JP)

(72) Inventors: Yumi HOSHINA, Tokyo (JP); Taro KUMAGAI, Tokyo (JP)

(73) Assignee: Mitsubishi Electric Mobility Corporation, Tokyo (JP)

(21) Appl. No.: 19/159,129

(22) PCT Filed: May 29, 2023
(86) PCT No.: PCT/JP2023/019830

§ 371 Date: Aug. 22, 2025

(51) Int. Cl. B60K 28/06; A61B 5/00; G06V 10/10; G06V 40/20

(52) U.S. Cl. CPC B60K 28/066; A61B 5/4845; G06V 10/10; G06V 40/23

(57) ABSTRACT

An alcohol intake determining device includes: a video image acquiring unit to acquire a captured video image of an occupant in a vehicle; a beverage intake behavior determining unit to determine whether the occupant has performed an action of taking a beverage in the vehicle, on the basis of the captured video image acquired by the video image acquiring unit; a vital information acquiring unit to acquire vital information about the occupant; and a drunken state determining unit to determine whether the occupant is under the influence of alcohol on the basis of the vital information acquired by the vital information acquiring unit, when the beverage intake behavior determining unit determines that an action of taking a beverage has been performed.

### TECHNICAL FIELD

The present disclosure relates to an alcohol intake determining device and an alcohol intake determining method.

### BACKGROUND ART

There are alcohol intake determining devices that determine whether an occupant in a vehicle is under the influence of alcohol (hereinafter, this state will be referred to as a “drunken state”).

As such an alcohol intake determining device, Patent Literature 1 discloses a monitoring device that acquires a captured video image of an occupant in a vehicle from a camera that captures the video image of the occupant, and determines whether the occupant is in a drunken state, on the basis of the facial complexion of the occupant in the captured video image or the body temperature of the occupant in the captured video image.

### CITATION LIST

### Patent Literature

Patent Literature 1: JP 2010-186251 A

### SUMMARY OF INVENTION

### Technical Problem

Since the monitoring device disclosed in Patent Literature 1 detects that an occupant is in a drunken state on the basis of a captured video image from a camera, there is a problem in that a drunken state of an occupant might be erroneously detected. The facial complexions and body temperatures vary among occupants. Therefore, even if an occupant has not drunk alcohol, the face of the occupant might turn red, and, even if an occupant has not drunk alcohol, the body temperature of the occupant might rise. In such cases, a drunken state of an occupant might be erroneously detected.

The present disclosure has been made to solve the above problem, and aims to obtain an alcohol intake determining device that can reduce erroneous detection of a drunken state more effectively than the monitoring device disclosed in Patent Literature 1.

### Solution to Problem

An alcohol intake determining device according to the present disclosure includes: a video image acquiring unit to acquire a captured video image of an occupant in a vehicle; a beverage intake behavior determining unit to determine whether the occupant has performed an action of taking a beverage in the vehicle, on the basis of the captured video image acquired by the video image acquiring unit; a vital information acquiring unit to acquire vital information about the occupant; and a drunken state determining unit to determine whether the occupant is under the influence of alcohol on the basis of the vital information acquired by the vital information acquiring unit, when the beverage intake behavior determining unit determines that an action of taking a beverage has been performed.

### Advantageous Effects of Invention

According to the present disclosure, it is possible to reduce erroneous detection of a drunken state more effectively than the monitoring device disclosed in Patent Literature 1.

### BRIEF DESCRIPTION OF DRAWINGS

FIG. 1 is a configuration diagram illustrating an alcohol intake determining device 3 according to a first embodiment.

FIG. 2 is a hardware configuration diagram illustrating the hardware of the alcohol intake determining device 3 according to the first embodiment.

FIG. 3 is a hardware configuration diagram of a computer in a case where the alcohol intake determining device 3 is formed with software, firmware, or the like.

FIG. 4 is a flowchart showing an alcohol intake determining method as procedures to be carried out by the alcohol intake determining device 3.

FIG. 5A is an explanatory diagram illustrating a state in which an occupant puts the rim of a container to his/her mouth, and starts drinking alcohol. FIG. 5B is an explanatory diagram illustrating a state in which the occupant is drinking alcohol while keeping the rim of the container to his/her mouth.

FIG. 6 is a configuration diagram illustrating an alcohol intake determining device 3 according to a second embodiment.

FIG. 7 is a hardware configuration diagram illustrating the hardware of the alcohol intake determining device 3 according to the second embodiment.

FIG. 8 is a configuration diagram illustrating an alcohol intake determining device 3 according to a third embodiment.

FIG. 9 is a hardware configuration diagram illustrating the hardware of the alcohol intake determining device 3 according to the third embodiment.

FIG. 10 is a configuration diagram illustrating an alcohol intake determining device 3 according to a fourth embodiment.

FIG. 11 is a hardware configuration diagram illustrating the hardware of the alcohol intake determining device 3 according to the fourth embodiment.

### DESCRIPTION OF EMBODIMENTS

To explain the present disclosure in greater detail, embodiments for carrying out the disclosure are described below with reference to the accompanying drawings.

### First Embodiment

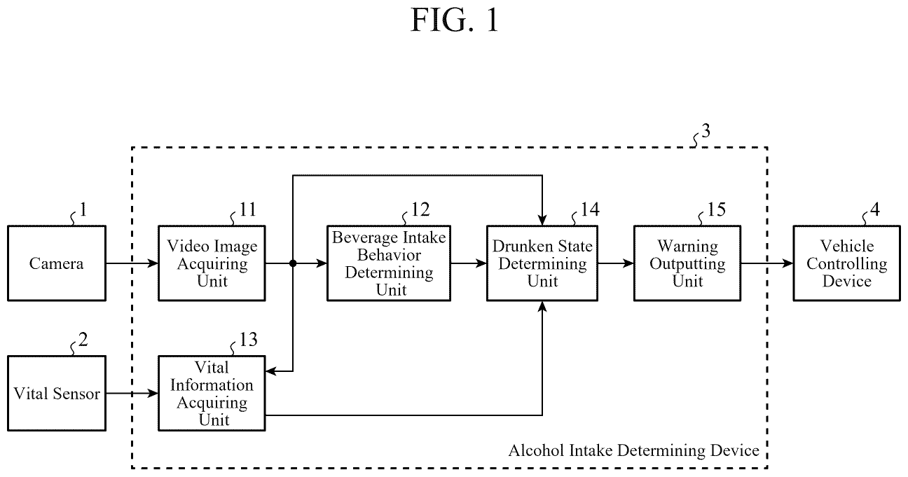

FIG. 1 is a configuration diagram illustrating an alcohol intake determining device 3 according to a first embodiment.

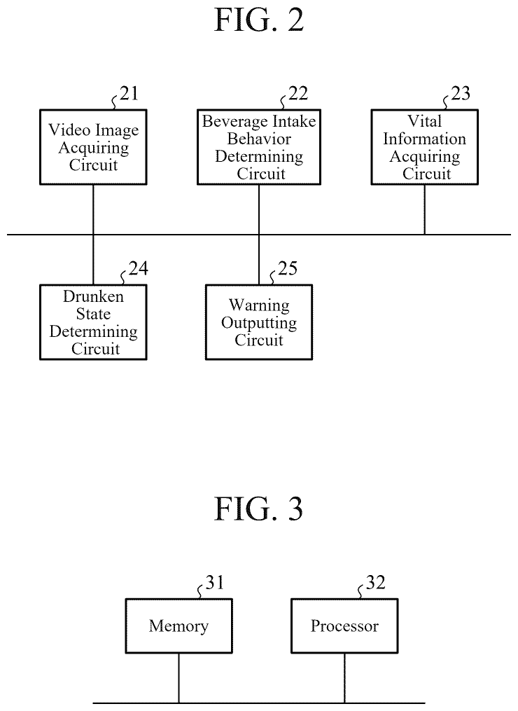

FIG. 2 is a hardware configuration diagram illustrating the hardware of the alcohol intake determining device 3 according to the first embodiment.

The alcohol intake determining device 3 illustrated in FIG. 1 includes a video image acquiring unit 11, a beverage intake behavior determining unit 12, a vital information acquiring unit 13, a drunken state determining unit 14, and a warning outputting unit 15.

A camera 1 is installed on the instrument panel, the ceiling, the A-pillar, or the windshield of a vehicle, for example.

The camera I captures a video image of an occupant in the vehicle, and transmits the captured video image of the occupant to the alcohol intake determining device 3.

The alcohol intake determining device 3 shown in FIG. 1 assumes that the occupant is the driver of the vehicle. Note that the occupant is not necessarily the driver of the vehicle, and may be an occupant other than the driver.

A vital sensor 2 is installed in the vehicle cabin, for example. Note that this is merely an example, and the vital sensor 2 may be attached to a wrist or the like of the occupant.

The vital sensor 2 measures vitals such as the heart rate of the occupant, and transmits vital information indicating the vitals of the occupant to the alcohol intake determining device 3.

The vital sensor 2 installed in the vehicle cabin measures the vitals of the occupant in a state of non-contact with the occupant. The vital sensor 2 captures a video image of the occupant with a built-in camera, for example, and measures the vitals of the occupant on the basis of the captured video image of the occupant.

The vital sensor 2 attached to a wrist or the like of the occupant directly measures the heart rate of the occupant. For this reason, the vital sensor 2 attached to a wrist or the like of the occupant generally has a higher measurement accuracy than the vital sensor 2 installed in the vehicle cabin.

The video image acquiring unit 11 is formed with a video image acquiring circuit 21 shown in FIG. 2, for example.

The video image acquiring unit 11 acquires the captured video image of the occupant in the vehicle from the camera 1.

The video image acquiring unit 11 outputs the captured video image of the occupant to each of the beverage intake behavior determining unit 12, the vital information acquiring unit 13, and the drunken state determining unit 14.

The beverage intake behavior determining unit 12 is formed with a beverage intake behavior determining circuit 22 shown in FIG. 2, for example.

The beverage intake behavior determining unit 12 acquires the captured video image of the occupant from the video image acquiring unit 11.

The beverage intake behavior determining unit 12 determines whether the occupant has performed an action of taking a beverage in the vehicle, on the basis of the captured video image.

The beverage intake behavior determining unit 12 outputs a determination result indicating whether an action of taking a beverage has been performed to the drunken state determining unit 14.

The vital information acquiring unit 13 is formed with a vital information acquiring circuit 23 shown in FIG. 2, for example.

The vital information acquiring unit 13 acquires, from the vital sensor 2, vital information indicating the vitals of the occupant.

The vital information acquiring unit 13 outputs the vital information to the drunken state determining unit 14.

In the alcohol intake determining device 3 illustrated in FIG. 1, the vital information acquiring unit 13 acquires the vital information from the vital sensor 2. Note that this is merely an example, and the vital information acquiring unit 13 may acquire the captured video image from the video image acquiring unit 11. In this case, the vital information acquiring unit 13 acquires the pulse rate of the occupant, the heart rate of the occupant, or the like as the vital information about the occupant on the basis of the captured video image, and outputs the vital information indicating the pulse rate or the like of the occupant to the drunken state determining unit 14.

The drunken state determining unit 14 is formed with a drunken state determining circuit 24 shown in FIG. 2, for example.

The drunken state determining unit 14 acquires a determination result from the beverage intake behavior determining unit 12, and acquires the vital information from the vital information acquiring unit 13.

When the beverage intake behavior determining unit 12 determines that an action of taking a beverage has been performed, the drunken state determining unit 14 determines whether the occupant is under the influence of alcohol (hereinafter referred to as a “drunken state”), on the basis of the vital information.

The drunken state determining unit 14 outputs a determination result indicating whether the occupant is in a drunken state, to the warning outputting unit 15.

In the alcohol intake determining device 3 shown in FIG. 1, the drunken state determining unit 14 determines whether the occupant is in a drunken state, on the basis of the vital information. Note that this is merely an example, and the drunken state determining unit 14 may determine whether the occupant is in a drunken state, on the basis of the captured video image acquired by the video image acquiring unit 11.

The warning outputting unit 15 is formed with a warning outputting circuit 25 shown in FIG. 2, for example.

The warning outputting unit 15 acquires, from the drunken state determining unit 14, a determination result indicating whether the occupant is in a drunken state.

The warning outputting unit 15 outputs a warning to the occupant, when the drunken state determining unit 14 determines that the occupant is in a drunken state. Specifically, the warning outputting unit 15 outputs warning information to a vehicle controlling device 4, to cause the vehicle controlling device 4 to output a warning.

When obtaining the warning information from the warning outputting unit 15, the vehicle controlling device 4 causes the instrument panel of the vehicle to display the warning, for example.

When obtaining the warning information from the warning outputting unit 15, the vehicle controlling device 4 controls the vehicle in such a manner that the vehicle will stop after moving to a road shoulder, for example.

In FIG. 1, it is assumed that each of the video image acquiring unit 11, the beverage intake behavior determining unit 12, the vital information acquiring unit 13, the drunken state determining unit 14, and the warning outputting unit 15, which are components of the alcohol intake determining device 3, is formed with dedicated piece of hardware as illustrated in FIG. 2. That is, it is assumed that the alcohol intake determining device 3 is formed with the video image acquiring circuit 21, the beverage intake behavior determining circuit 22, the vital information acquiring circuit 23, the drunken state determining circuit 24, and the warning outputting circuit 25.

Each of the video image acquiring circuit 21, the beverage intake behavior determining circuit 22, the vital information acquiring circuit 23, the drunken state determining circuit 24, and the warning outputting circuit 25 is a single circuit, a composite circuit, a programmed processor, a parallel-programmed processor, an application specific integrated circuit (ASIC), a field-programmable gate array (FPGA), or a combination thereof, for example.

The components of the alcohol intake determining device 3 are not necessarily formed with dedicated hardware, but the alcohol intake determining device 3 may be formed with software, firmware, or a combination of software and firmware.

Software or firmware is stored as a program in a memory of a computer. A computer means hardware that executes a program, and is a central processing unit (CPU), a graphics processing unit (GPU), a central processor, a processing unit, an arithmetic unit, a microprocessor, a microcomputer, a processor, or a digital signal processor (DSP), for example.

FIG. 3 is a hardware configuration diagram of a computer in a case where the alcohol intake determining device 3 is formed with software, firmware, or the like.

In a case where the alcohol intake determining device 3 is formed with software, firmware, or the like, a program for causing a computer to carry out each of the processing procedures in the video image acquiring unit 11, the beverage intake behavior determining unit 12, the vital information acquiring unit 13, the drunken state determining unit 14, and the warning outputting unit 15 is stored in a memory 31. A processor 32 of the computer then executes the program stored in the memory 31.

Further, FIG. 2 illustrates an example in which each of the components of the alcohol intake determining device 3 is formed with dedicated hardware, and FIG. 3 illustrates an example in which the alcohol intake determining device 3 is formed with software, firmware, or the like. Note that this is merely an example, and some components in the alcohol intake determining device 3 may be formed with dedicated hardware while the remaining components are formed with software, firmware, or the like.

Next, an operation to be performed by the alcohol intake determining device 3 illustrated in FIG. 1 is described.

The camera 1 captures a video image of an occupant in the vehicle, and transmits the captured video image of the occupant to the alcohol intake determining device 3. The captured video image of the occupant is not necessarily a moving image, but may be a plurality of still images arranged in time series.

The vital sensor 2 measures vitals such as the heart rate of the occupant, and transmits vital information indicating the vitals of the occupant to the alcohol intake determining device 3.

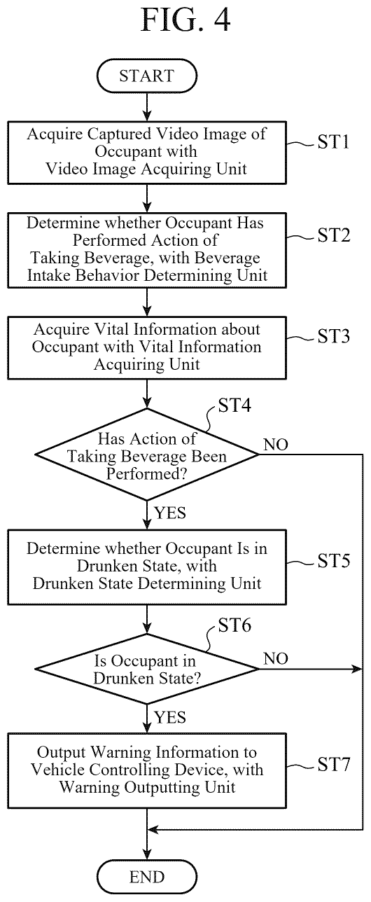

FIG. 4 is a flowchart showing an alcohol intake determining method as procedures to be carried out by the alcohol intake determining device 3.

The video image acquiring unit 11 acquires a captured video image of an occupant from the camera 1 (step ST1 in FIG. 4).

The video image acquiring unit 11 outputs the captured video image of the occupant to the beverage intake behavior determining unit 12.

When the vital information acquiring unit 13 uses the captured video image of the occupant to acquire vital information, the video image acquiring unit 11 also outputs the captured video image to the vital information acquiring unit 13.

When the drunken state determining unit 14 uses the captured video image of the occupant to determine a drunken state, the video image acquiring unit 11 also outputs the captured video image to the drunken state determining unit 14.

The beverage intake behavior determining unit 12 acquires the captured video image of the occupant from the video image acquiring unit 11.

The beverage intake behavior determining unit 12 determines whether the occupant has performed an action of taking a beverage in the vehicle, on the basis of the captured video image (step ST2 in FIG. 4).

The beverage intake behavior determining unit 12 outputs a determination result indicating whether an action of taking a beverage has been performed to the drunken state determining unit 14.

In the following, a process to be performed by the beverage intake behavior determining unit 12 to determine an action of taking a beverage is specifically described.

The beverage intake behavior determining unit 12 analyzes the captured video image of the occupant, to perform a process of detecting a state in which a hand of the occupant is holding a container such as a cup, a glass, a can, or a glass bottle. The process of detecting a state in which a hand of the occupant is holding a container is a known technique, and therefore, detailed explanation thereof is not made herein.

When detecting a state in which a hand of the occupant is holding a container, the beverage intake behavior determining unit 12 detects, from the captured video image of the occupant, a change in the position of the head of the occupant, a change in the position of the hand of the occupant, and a change in the angle of the head of the occupant.

Here, the head of the occupant is a portion above the neck of the occupant, and includes the entire face of the occupant.

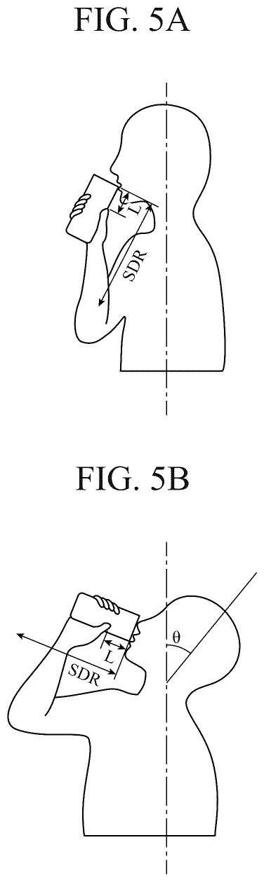

As illustrated in FIG. 5, the beverage intake behavior determining unit 12 detects whether the distance between the position of the head and the position of the hand is within a set distance range SDR, on the basis of a change in the position of the head and a change in the position of the hand. In the example in FIG. 5, the beverage intake behavior determining unit 12 detects a change in the position of the mouth as the position of the head.

FIG. 5A is an explanatory diagram illustrating a state in which an occupant puts the rim of a container to his/her mouth, and starts drinking alcohol.

FIG. 5B is an explanatory diagram illustrating a state in which the occupant is drinking alcohol while keeping the rim of the container to his/her mouth.

If the distance L between the position of the mouth of the occupant and the position of the hand is out of the set distance range SDR, the possibility that the occupant will drink alcohol is low. If the distance L between the position of the mouth of the occupant and the position of the hand is within the set distance range SDR, on the other hand, the possibility that the occupant will drink alcohol is high.

The set distance range SDR is determined depending on the size of the container. For example, the minimum value of the set distance range SDR is determined to be 0 [cm], and the maximum value of the set distance range SDR is determined to be a value that is greater than the height of the container by several tens of cm. The set distance range SDR may be stored in an internal memory of the beverage intake behavior determining unit 12, or may be given from the outside the alcohol intake determining device 3.

In a state where the occupant is drinking alcohol after the occupant puts the rim of the container to his/her mouth in order for the occupant to drink alcohol, the distance L between the position of the mouth and the position of the hand hardly changes, as illustrated in FIGS. 5A and 5B.

When an occupant actually drinks alcohol, the angle θ of the head often changes in such a manner that the occupant faces upward, as illustrated in FIG. 5B.

When the distance L between the position of the mouth and the position of the hand falls within the set distance range SDR, there is a high possibility that the occupant will drink alcohol. Therefore, as illustrated in FIGS. SA and 5B, the beverage intake behavior determining unit 12 determines whether the angle θ of the head has changed in such a manner that the occupant faces upward when the distance L between the position of the mouth and the position of the hand hardly changes.

A check can be made to determine whether the distance L is hardly changing, by checking whether a state in which the change ΔL in the distance L is equal to or smaller than a threshold Lₜₕ is maintained. The threshold Lₜₕ may be stored in an internal memory of the beverage intake behavior determining unit 12, or may be given from the outside the alcohol intake determining device 3.

As the distance L between the position of the mouth and the position of the hand, the distance between the position of the lower lip of the occupant and the position of the thumb can be used, for example. The position of the lower lip may be the position of the center of the lower lip, or may be the position of an end of the lower lip, for example. The position of the thumb may be the position of the tip of the thumb, the position of the joint of the thumb, or the position of the base of the thumb, for example.

The angle θ of the head is the angle formed by the vertical direction and the line segment connecting the center of the neck and the center of the top of the head. The process of calculating the angle θ of the head on the basis of a captured video image of the occupant is a known technique, and therefore, detailed explanation thereof is not made herein.

In the example in FIG. 5A, the angle θ of the head of the occupant at the start of drinking is 0 degrees.

In the example in FIG. 5B, the angle θ of the head is greater than 0 degrees, because the head is inclined in such a manner that the occupant faces upward to drink alcohol.

When the angle θ of the head changes in such a manner that the occupant faces upward in a state where the distance L between the position of the mouth and the position of the hand hardly changes, the beverage intake behavior determining unit 12 outputs a determination result indicating that an action of taking a beverage has been performed, to the drunken state determining unit 14.

In a case where a state in which the distance L between the position of the mouth and the position of the hand hardly changes is not detected, or in a case where the angle θ of the head has not changed in such a manner that the occupant faces upward in the state even when the state in which the distance L hardly changes, the beverage intake behavior determining unit 12 outputs a determination result indicating that an action of taking a beverage has not been performed, to the drunken state determining unit 14.

In the alcohol intake determining device 3 illustrated in FIG. 1, when a state in which the hand of the occupant is holding a container, the beverage intake behavior determining unit 12 detects a change in the position of the head of the occupant, a change in the position of the hand of the occupant, and a change in the angle of the head of the occupant from the captured video image of the occupant, and determines whether the occupant has performed an action of taking a beverage, on the basis of the change in the position of the head, the change in the position of the hand, and the change in the angle of the head. However, when the container is small, the container may be hidden by the hand in a state where the hand of the occupant is holding the container. In such a case, there is a possibility that the state in which the hand of the occupant is holding the container cannot be detected. Therefore, even when the state in which the hand of the occupant is holding the container cannot be detected, the beverage intake behavior determining unit 12 may determine whether the occupant has performed an action of taking a beverage, on the basis of a change in the position of the head, a change in the position of the hand, and a change in the angle of the head.

In the alcohol intake determining device 3 illustrated in FIG. 1, the beverage intake behavior determining unit 12 determines whether the occupant has performed an action of taking a beverage, on the basis of a change in the position of the head, a change in the position of the hand, and a change in the angle of the head. However, there also is a possibility that the occupant drinks alcohol with a straw, and therefore, the beverage intake behavior determining unit 12 may determine that the occupant bas performed an action of taking a beverage when detecting, from a captured video image of the occupant, a state in which the occupant puts his/her mouth to the straw.

In the alcohol intake determining device 3 illustrated in FIG. 1, the beverage intake behavior determining unit 12 determines whether the occupant has performed an action of taking a beverage, on the basis of a change in the position of the head, a change in the position of the hand, and a change in the angle of the head. Note that this is merely an example, and the beverage intake behavior determining unit 12 may determine that the occupant has performed an action of taking a beverage, when detecting, from a captured video image of the occupant, that part of the mouth of the occupant is hidden by the container.

In the alcohol intake determining device 3 illustrated in FIG. 1, the beverage intake behavior determining unit 12 determines whether the occupant has performed an action of taking a beverage, on the basis of a captured video image of the occupant. When a sensor signal from a sensor (not shown) that detects the smell of alcohol indicates that the smell of alcohol is detected, the beverage intake behavior determining unit 12 may determine whether the occupant has performed an action of taking a beverage, on the basis of a captured video image of the occupant. In this manner, in a case where a sensor signal from a sensor that detects the smell of alcohol is used, the accuracy of determination by the beverage intake behavior determining unit 12 can be enhanced.

Also, the beverage intake behavior determining unit 12 may supply a captured video image of the occupant to a first trained model (not shown), and acquire, from the first trained model, a determination result indicating whether an action of taking a beverage has been performed.

The first trained model is formed with a neural network, for example. The first trained model is supplied with first learning data and second learning data at the time of learning. The first learning data includes a video image captured when the occupant is performing an action of taking a beverage, and training data indicating that the occupant is performing an action of taking a beverage. The second learning data includes a video image captured when the occupant is not performing an action of taking a beverage, and training data indicating that the occupant is not performing an action of taking a beverage.

Supplied with the first learning data, the first trained model learns the video image captured when the occupant is performing an action of taking a beverage. Supplied with the second learning data, the first trained model learns the video image captured when the occupant is not performing an action of taking a beverage.

Supplied with the captured video image acquired by the video image acquiring unit 11 at the time of inference, the first trained model outputs a determination result indicating whether an action of taking a beverage has been performed.

The vital information acquiring unit 13 acquires, from the vital sensor 2, vital information indicating the vitals of the occupant (step ST3 in FIG. 4).

The vital information acquiring unit 13 outputs the vital information to the drunken state determining unit 14.

In the alcohol intake determining device 3 illustrated in FIG. 1, the vital information acquiring unit 13 acquires the vital information from the vital sensor 2. Note that this is merely an example, and the vital information acquiring unit 13 may acquire the captured video image from the video image acquiring unit 11. In this case, the vital information acquiring unit 13 acquires the pulse rate of the occupant, the heart rate of the occupant, or the like as the vital information about the occupant on the basis of the captured video image, and outputs the vital information indicating the pulse rate or the like of the occupant to the drunken state determining unit 14. The process of measuring vitals such as a pulse rate on the basis of a captured video image is a known technique, and therefore, detailed explanation thereof is not made herein.

The drunken state determining unit 14 acquires a determination result from the beverage intake behavior determining unit 12, and acquires the vital information from the vital information acquiring unit 13.

If the beverage intake behavior determining unit 12 determines that an action of taking a beverage has been performed (step ST4 in FIG. 4: YES), the drunken state determining unit 14 determines whether the occupant is in a drunken state, on the basis of the vital information (step ST5 in FIG. 4).

In the following, a process to be performed by the drunken state determining unit 14 to determine a drunken state is specifically described.

In a memory in the drunken state determining unit 14, the heart rate or the pulse rate of the occupant not in a drunken state is recorded. As the heart rate or the like of the occupant not in a drunken state, the heart rate indicated by the vital information output from the vital information acquiring unit 13 when the occupant not in a drunken state gets in the vehicle can be used, for example.

When the beverage intake behavior determining unit 12 determines that an action of taking a beverage has been performed, the drunken state determining unit 14 acquires the vital information from the vital information acquiring unit 13.

The drunken state determining unit 14 then compares the heart rate or the like indicated by the vital information with the heart rate or the like recorded in the memory.

The drunken state determining unit 14 determines whether the occupant is in a drunken state, on the basis of the result of the comparison between the heart rates or the like. The process of determining a drunken state on the basis of a result of comparison between heart rates or the like is a known technique, and therefore, detailed explanation thereof is not made herein. In general, it is known that the heart rate or the like increases as the influence of alcohol progresses with drinking.

In the alcohol intake determining device 3 illustrated in FIG. 1, the drunken state determining unit 14 determines whether the occupant is in a drunken state, on the basis of a result of comparison between heart rates or the like. Note that this is merely an example, and the drunken state determining unit 14 may supply the vital information about the occupant to a second trained model (not shown), and acquire a result of determination as to a drunken state from the second trained model.

The second trained model is formed with a neural network, for example. The second trained model is supplied with third learning data and fourth learning data at the time of learning. The third learning data includes the vital information at a time when the occupant has drunk alcohol, and training data indicating that the occupant has drunk alcohol. The fourth learning data includes the vital information at a time when the occupant has not drunk alcohol, and training data indicating that the occupant has not drunk alcohol.

Supplied with the third learning data, the second trained model learns the vital information at the time when the occupant has drunk alcohol. Supplied with the second learning data, the second trained model learns the vital information at the time when the occupant has not drunk alcohol.

Supplied with vital information at the time of inference, the second trained model outputs a result of drunken state determination.

In the alcohol intake determining device 3 illustrated in FIG. 1, the drunken state determining unit 14 determines whether the occupant is in a drunken state, on the basis of the vital information output from the vital information acquiring unit 13. Note that this is merely an example, and the drunken state determining unit 14 may determine whether the occupant is in a drunken state, on the basis of the captured video image acquired by the video image acquiring unit 11. In this case, the drunken state determining unit 14 detects the number of times the occupant looks down within a certain period of time, by detecting changes in the position of the head of the occupant on the basis of the captured video image of the occupant. Also, the drunken state determining unit 14 detects the number of times the occupant yawns within a certain period of time, on the basis of the captured video image of the occupant. The drunken state determining unit 14 may then determine that the occupant is in a drunken state, when the number of times the occupant looks down and/or the number of times the occupant yawns exceeds a reference number.

Here, the drunken state determining unit 14 determines whether the occupant is in a drunken state, on the basis of the captured video image acquired by the video image acquiring unit 11. Note that this is merely an example, and the drunken state determining unit 14 may supply the captured video image of the occupant to a third trained model (not shown), and acquire a result of determination as to a drunken state from the third trained model.

The third trained model is formed with a neural network, for example. The third trained model is supplied with fifth learning data and sixth learning data at the time of learning. The fifth learning data includes a video image captured when the occupant has drunk alcohol, and training data indicating that the occupant has drunk alcohol. The sixth learning data includes a video image captured when the occupant has not drunk alcohol, and training data indicating that the occupant has not drunk alcohol.

Supplied with the fifth learning data, the third trained model learns the video image captured when the occupant has drunk alcohol. Supplied with the sixth learning data, the third trained model learns the video image captured when the occupant has not drunk alcohol.

Supplied with the captured video image acquired by the video image acquiring unit 11 at the time of inference, the third trained model outputs a result of determination as to a drunken state.

The drunken state determining unit 14 outputs a determination result indicating whether the occupant is in a drunken state, to the warning outputting unit 15.

In the alcohol intake determining device 3 illustrated in FIG. 1, if the beverage intake behavior determining unit 12 determines that an action of taking a beverage has not been performed (step ST4 in FIG. 4: NO), the drunken state determining unit 14 does not perform a process of determining whether the occupant is in a drunken state.

The beverage intake behavior determining unit 12 determines whether the occupant is in a drunken state only when an action of taking a beverage has been performed, and thus, it is possible to prevent the occurrence of erroneous determination to the effect that the occupant is in a drunken state even though any action of taking a beverage has not been performed.

When the beverage intake behavior determining unit 12 does not determine that an action of taking a beverage has been performed, the drunken state determining unit 14 does not perform the process of determining whether the occupant is under the influence of alcohol, and thus, the processing load on the alcohol intake determining device 3 can be reduced.

The warning outputting unit 15 acquires, from the drunken state determining unit 14, a determination result indicating whether the occupant is in a drunken state.

If the drunken state determining unit 14 determines that the occupant is in a drunken state (step ST6 in FIG. 4: YES), the warning outputting unit 15 outputs warning information to the vehicle controlling device 4, to cause the vehicle controlling device 4 to output a warning.

If the drunken state determining unit 14 determines that the occupant is not in a drunken state (step ST6 in FIG. 4: NO), the warning outputting unit 15 does not output warning information to the vehicle controlling device 4.

When the warning information is output from the warning outputting unit 15, the vehicle controlling device 4 causes the instrument panel of the vehicle to display the warning, for example.

When obtaining the warning information from the warning outputting unit 15, the vehicle controlling device 4 controls the vehicle in such a manner that the vehicle will stop after moving to a road shoulder, for example.

In the first embodiment described above, the alcohol intake determining device 3 includes: the video image acquiring unit 11 that acquires a captured video image of an occupant in a vehicle; the beverage intake behavior determining unit 12 that determines whether the occupant has performed an action of taking a beverage in the vehicle, on the basis of the captured video image acquired by the video image acquiring unit 11; the vital information acquiring unit 13 that acquires vital information about the occupant; and the drunken state determining unit 14 that determines whether the occupant is under the influence of alcohol, on the basis of the vital information acquired by the vital information acquiring unit 13, when the beverage intake behavior determining unit 12 determines that an action of taking a beverage has been performed. Thus, the alcohol intake determining device 3 can reduce erroneous detection of a drunken state more effectively than the monitoring device disclosed in Patent Literature 1.

### Second Embodiment

In a second embodiment, an alcohol intake determining device 3 including a vehicle information acquiring unit 16 that acquires vehicle information indicating a steering angle of the steering wheel of a vehicle is described.

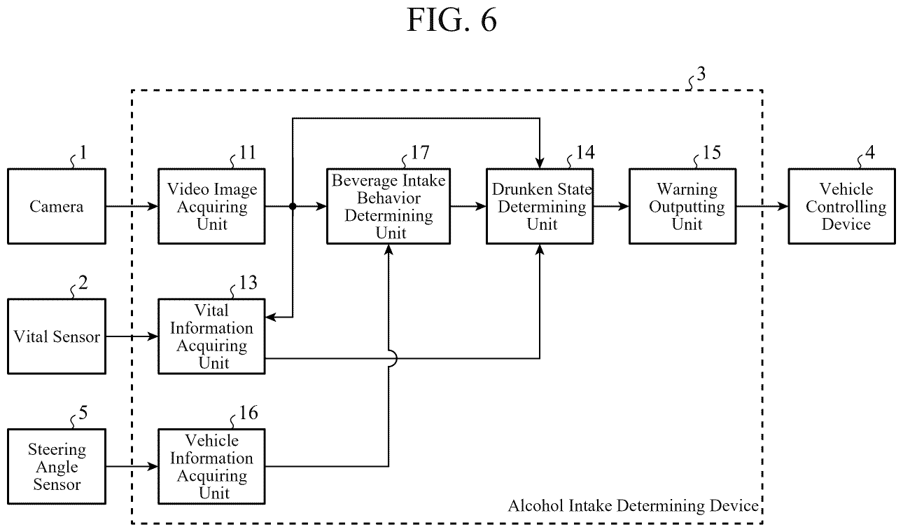

FIG. 6 is a configuration diagram illustrating the alcohol intake determining device 3 according to the second embodiment. In FIG. 6, the same reference numerals as those in FIG. 1 denote the same or corresponding components, and therefore, detailed explanation of them is not made herein.

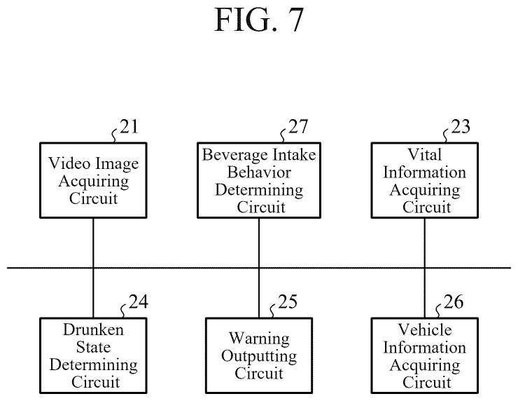

FIG. 7 is a hardware configuration diagram illustrating the hardware of the alcohol intake determining device 3 according to the second embodiment. In FIG. 7, the same reference numerals as those in FIG. 2 denote the same or corresponding components, and therefore, detailed explanation of them is not made herein.

The alcohol intake determining device 3 illustrated in FIG. 6 includes a video image acquiring unit 11, the vehicle information acquiring unit 16, a beverage intake behavior determining unit 17, a vital information acquiring unit 13, a drunken state determining unit 14, and a warning outputting unit 15.

A steering angle sensor 5 measures the steering angle of the steering wheel of the vehicle, and outputs vehicle information indicating the steering angle of the steering wheel to the alcohol intake determining device 3.

The vehicle information acquiring unit 16 is formed with a vehicle information acquiring circuit 26 shown in FIG. 7, for example.

The vehicle information acquiring unit 16 acquires the vehicle information indicating the steering angle of the steering wheel of the vehicle from the steering angle sensor 5. Note that the vehicle information is not necessarily acquired from the steering angle sensor 5, but may be acquired from the vehicle controlling device 4, for example.

The vehicle information acquiring unit 16 outputs the vehicle information to beverage intake behavior determining unit 17.

The beverage intake behavior determining unit 17 is formed with a beverage intake behavior determining circuit 27 shown in FIG. 7, for example.

The beverage intake behavior determining unit 17 acquires a captured video image of the occupant from the video image acquiring unit 11, and acquires the vehicle information from the vehicle information acquiring unit 16.

Only when the steering angle indicated by the vehicle information is equal to or smaller than a threshold, does the beverage intake behavior determining unit 17 determine whether the occupant has performed an action of taking a beverage in the vehicle, on the basis of the captured video image. The threshold may be stored in an internal memory of the beverage intake behavior determining unit 17, or may be given from the outside the alcohol intake determining device 3.

The beverage intake behavior determining unit 17 outputs a determination result indicating whether an action of taking a beverage has been performed to the drunken state determining unit 14.

In FIG. 6, it is assumed that each of the video image acquiring unit 11, the vehicle information acquiring unit 16, the beverage intake behavior determining unit 17, the vital information acquiring unit 13, the drunken state determining unit 14, and the warning outputting unit 15, which are components of the alcohol intake determining device 3, is formed with dedicated piece of hardware as illustrated in FIG. 7. That is, it is assumed that the alcohol intake determining device 3 is formed with the video image acquiring circuit 21, the vehicle information acquiring circuit 26, the beverage intake behavior determining circuit 27, the vital information acquiring circuit 23, the drunken state determining circuit 24, and the warning outputting circuit 25,

Each of the video image acquiring circuit 21, the vehicle information acquiring circuit 26, the beverage intake behavior determining circuit 27, the vital information acquiring circuit 23, the drunken state determining circuit 24, and the warning outputting circuit 25 is a single circuit, a composite circuit, a programmed processor, a parallel-programmed processor, an ASIC, an FPGA, or a combination thereof, for example.

The components of the alcohol intake determining device 3 are not necessarily formed with dedicated hardware, but the alcohol intake determining device 3 may be formed with software, firmware, or a combination of software and firmware.

In a case where the alcohol intake determining device 3 is formed with software, firmware, or the like, a program for causing a computer to carry out each of the processing procedures in the video image acquiring unit 11, the vehicle information acquiring unit 16, the beverage intake behavior determining unit 17, the vital information acquiring unit 13, the drunken state determining unit 14, and the warning outputting unit 15 is stored in the memory 31 shown in FIG. 3. The processor 32 shown in FIG. 3 then executes the program stored in the memory 31.

Further, FIG. 7 illustrates an example in which each of the components of the alcohol intake determining device 3 is formed with dedicated hardware, and FIG. 3 illustrates an example in which the alcohol intake determining device 3 is formed with software, firmware, or the like. Note that this is merely an example, and some components in the alcohol intake determining device 3 may be formed with dedicated hardware while the remaining components are formed with software, firmware, or the like.

Next, an operation to be performed by the alcohol intake determining device 3 illustrated in FIG. 6 is described. Note that, other than the vehicle information acquiring unit 16 and the beverage intake behavior determining unit 17, the alcohol intake determining device 3 is the same as the alcohol intake determining device 3 illustrated in FIG. 1. Therefore, operations to be performed by the vehicle information acquiring unit 16 and the beverage intake behavior determining unit 17 are mainly described herein.

The steering angle sensor 5 measures the steering angle of the steering wheel of the vehicle.

The steering angle sensor 5 outputs vehicle information indicating the steering angle of the steering wheel to the alcohol intake determining device 3.

The vehicle information acquiring unit 16 acquires the vehicle information from the steering angle sensor 5.

The vehicle information acquiring unit 16 outputs the vehicle information to beverage intake behavior determining unit 17.

The beverage intake behavior determining unit 17 acquires a captured video image of the occupant from the video image acquiring unit 11, and acquires the vehicle information from the vehicle information acquiring unit 16.

When the steering angle of the steering wheel is large, the traveling direction of the vehicle greatly changes. When the steering angle of the steering wheel is small, or particularly when the steering angle of the steering wheel is 0 degrees, on the other hand, the traveling direction of the vehicle does not change. Therefore, when the steering angle of the steering wheel is large, the occupant needs to concentrate on the driving operation of the vehicle more than when the steering angle of the steering wheel is small. Therefore, in a state where the steering angle of the steering wheel is large, the possibility that the occupant will drink alcohol is low.

Only when the steering angle indicated by the vehicle information is equal to or smaller than a threshold, does the beverage intake behavior determining unit 17 determine whether the occupant has performed an action of taking a beverage in the vehicle, on the basis of the captured video image. That is, only when the necessity for the occupant to concentrate on the vehicle driving operation is low, does the beverage intake behavior determining unit 17 determine whether the occupant has performed an action of taking a beverage in the vehicle, on the basis of the captured video image.

The beverage intake behavior determining unit 17 outputs a determination result indicating whether an action of taking a beverage has been performed to the drunken state determining unit 14.

In the second embodiment described above, the alcohol intake determining device 3 illustrated in FIG. 6 includes the vehicle information acquiring unit 16 that acquires vehicle information indicating the steering angle of the steering wheel of the vehicle, and the beverage intake behavior determining unit 17 determines whether the occupant has performed an action of taking a beverage in the vehicle only when the steering angle indicated by the vehicle information acquired by the vehicle information acquiring unit 16 is equal to or smaller than the threshold. Therefore, like the alcohol intake determining device 3 illustrated in FIG. 1, the alcohol intake determining device 3 illustrated in FIG. 6 can reduce erroneous detection of a drunken state more effectively than the monitoring device disclosed in Patent Literature 1, and can reduce the processing load on the process of determining an action of taking a beverage more effectively than the alcohol intake determining device 3 illustrated in FIG. 1.

### Third Embodiment

In an alcohol intake determining device 3 according to a third embodiment, a drunken state determining unit 18 determines whether an occupant is in a drunken state when a beverage intake behavior determining unit 12 has not determined that an action of taking a beverage has been performed, and determines that an abnormality has occurred in the occupant when the occupant is determined to be in a drunken state.

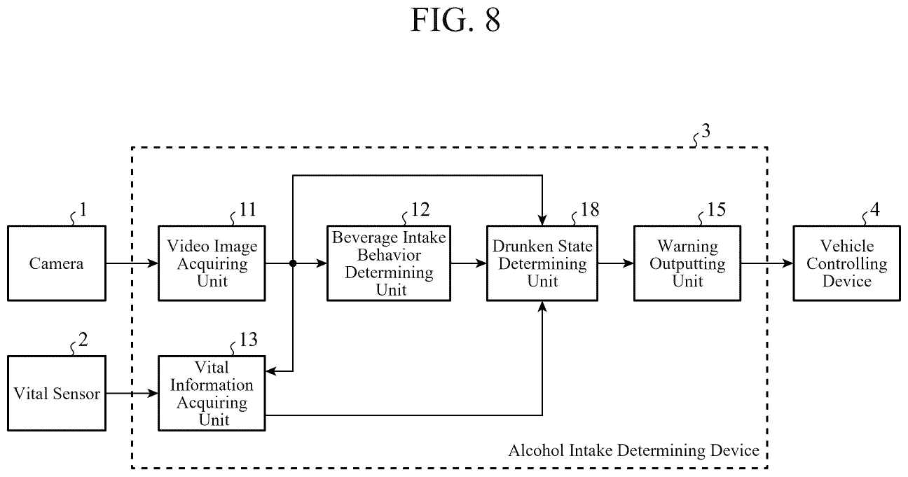

FIG. 8 is a configuration diagram illustrating the alcohol intake determining device 3 according to the third embodiment. In FIG. 8, the same reference numerals as those in FIG. 1 and FIG. 6 denote the same or corresponding components, and therefore, detailed explanation of them is not made herein.

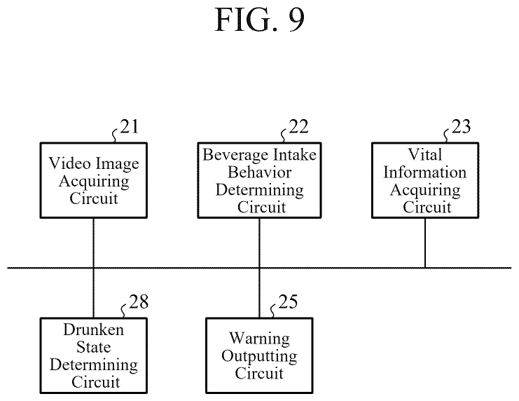

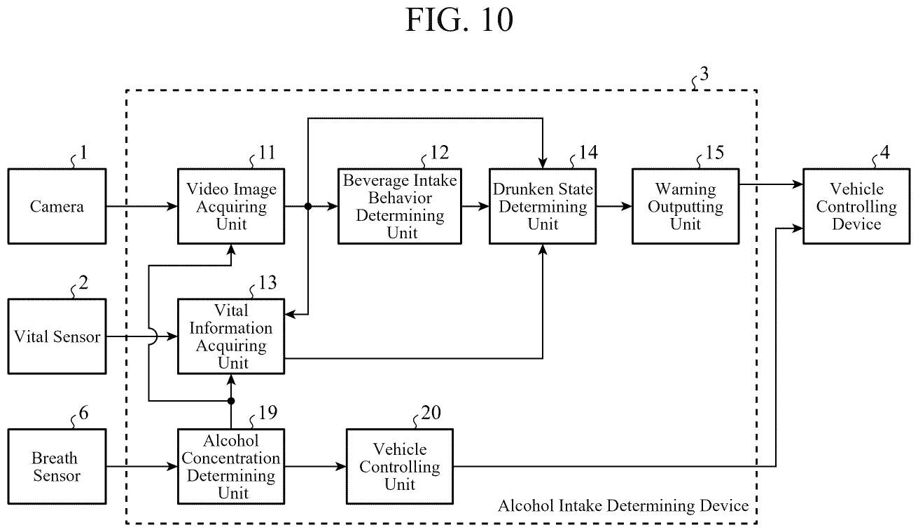

FIG. 9 is a hardware configuration diagram illustrating the hardware of the alcohol intake determining device 3 according to the third embodiment. In FIG. 9, the same reference numerals as those in FIG. 2 and FIG. 7 denote the same or corresponding components, and therefore, detailed explanation of them is not made herein.

The alcohol intake determining device 3 illustrated in FIG. 8 includes a video image acquiring unit 11, the beverage intake behavior determining unit 12, a vital information acquiring unit 13, the drunken state determining unit 18, and a warning outputting unit 15.

The drunken state determining unit 18 is formed with a drunken state determining circuit 28 shown in FIG. 9, for example.

The drunken state determining unit 18 acquires a determination result from the beverage intake behavior determining unit 12, acquires vital information from the vital information acquiring unit 13, and acquires a captured video image from the video image acquiring unit 11.

When the beverage intake behavior determining unit 12 has not determined that an action of taking a beverage has been performed, the drunken state determining unit 18 determines whether the occupant is in a drunken state on the basis of the vital information or the captured video image, like the drunken state determining unit 14 shown in FIG. 1.

When the beverage intake behavior determining unit 12 determines that an action of taking a beverage has not been performed, the drunken state determining unit 18 also determines whether the occupant is in a drunken state, unlike the drunken state determining unit 14 shown in FIG. 1. When determining that the occupant is in a drunken state, the drunken state determining unit 18 determines that an abnormality has occurred in the occupant.

The drunken state determining unit 18 outputs, to the warning outputting unit 15, a determination result indicating whether the occupant is in a drunken state, and a determination result indicating that an abnormality has occurred in the occupant.

The alcohol intake determining device 3 illustrated in FIG. 8 is the alcohol intake determining device 3 illustrated in FIG. 1, in which the drunken state determining unit 18 is adopted. Note that this is merely an example, and the drunken state determining unit 18 may be adopted in the alcohol intake determining device 3 illustrated in FIG. 6.

In FIG. 8, it is assumed that each of the video image acquiring unit 11, the beverage intake behavior determining unit 12, the vital information acquiring unit 13, the drunken state determining unit 18, and the warning outputting unit 15, which are components of the alcohol intake determining device 3, is formed with dedicated piece of hardware as illustrated in FIG. 9. That is, it is assumed that the alcohol intake determining device 3 is formed with the video image acquiring circuit 21, the beverage intake behavior determining circuit 22, the vital information acquiring circuit 23, the drunken state determining circuit 28, and the warning outputting circuit 25.

Each of the video image acquiring circuit 21, the beverage intake behavior determining circuit 22, the vital information acquiring circuit 23, the drunken state determining circuit 28, and the warning outputting circuit 25 is a single circuit, a composite circuit, a programmed processor, a parallel-programmed processor, an ASIC, an FPGA, or a combination thereof, for example.

The components of the alcohol intake determining device 3 are not necessarily formed with dedicated hardware, but the alcohol intake determining device 3 may be formed with software, firmware, or a combination of software and firmware.

In a case where the alcohol intake determining device 3 is formed with software, firmware, or the like, a program for causing a computer to carry out each of the processing procedures in the video image acquiring unit 11, the beverage intake behavior determining unit 12, the vital information acquiring unit 13, the drunken state determining unit 18, and the warning outputting unit 15 is stored in the memory 31 shown in FIG. 3. The processor 32 shown in FIG. 3 then executes the program stored in the memory 31.

Further, FIG. 9 illustrates an example in which each of the components of the alcohol intake determining device 3 is formed with dedicated hardware, and FIG. 3 illustrates an example in which the alcohol intake determining device 3 is formed with software, firmware, or the like. Note that this is merely an example, and some components in the alcohol intake determining device 3 may be formed with dedicated hardware while the remaining components are formed with software, firmware, or the like.

Next, an operation to be performed by the alcohol intake determining device 3 illustrated in FIG. 8 is described. The alcohol intake determining device 3 is the same as the alcohol intake determining device 3 illustrated in FIG. 1, except for the drunken state determining unit 18. Therefore, only an operation to be performed by the drunken state determining unit 18 is described herein.

The drunken state determining unit 18 acquires a determination result from the beverage intake behavior determining unit 12.

Further, the drunken state determining unit 18 acquires vital information from the vital information acquiring unit 13, and acquires a captured video image from the video image acquiring unit 11.

When the beverage intake behavior determining unit 12 determines that an action of taking a beverage has been performed, the drunken state determining unit 18 determines whether the occupant is in a drunken state on the basis of the vital information or the captured video image, like the drunken state determining unit 14 shown in FIG. 1.

The drunken state determining unit 18 outputs a determination result indicating whether the occupant is in a drunken state, to the warning outputting unit 15.

When the beverage intake behavior determining unit 12 has not determined that an action of taking a beverage has been performed, the drunken state determining unit 18 also determines whether the occupant is in a drunken state on the basis of the vital information or the captured video image, unlike the drunken state determining unit 14 shown in FIG. 1.

When an action of taking a beverage has not been performed, there is no possibility that the occupant will be in a drunken state. Therefore, determining that the occupant is in a drunken state when an action of taking a beverage has not been performed is highly likely erroneous determination due to occurrence of an abnormality other than a drunken state in the occupant.

When the occupant is determined to be in a drunken state when it has not been determined that an action of taking a beverage has been performed, the drunken state determining unit 18 determines that an abnormality has occurred in the occupant.

The drunken state determining unit 18 outputs, to the warning outputting unit 15, a determination result indicating that an abnormality has occurred in the occupant.

The warning outputting unit 15 acquires, from the drunken state determining unit 18, a determination result indicating whether the occupant is in a drunken state.

When the drunken state determining unit 18 determines that the occupant is in a drunken state, the warning outputting unit 15 outputs warning information to the vehicle controlling device 4, to cause the vehicle controlling device 4 to output a warning.

When a determination result indicating that an abnormality has occurred in the occupant is output from the drunken state determining unit 18, the warning outputting unit 15 outputs alert information indicating that an abnormality has occurred in the occupant to the vehicle controlling device 4, to cause the vehicle controlling device 4 to issue an alert.

In the third embodiment, the alcohol intake determining device 3 illustrated in FIG. 8 is designed so that the drunken state determining unit 18 determines whether an occupant is in a drunken state when the beverage intake behavior determining unit 12 has not determined that an action of taking a beverage has been performed, and determines that an abnormality has occurred in the occupant when the occupant is determined to be in a drunken state. Thus, like the alcohol intake determining device 3 illustrated in FIG. 1, the alcohol intake determining device 3 illustrated in FIG. 8 can reduce erroneous detection of a drunken state more effectively than the monitoring device disclosed in Patent Literature 1, and can alert the occupant having an abnormality.

### Fourth Embodiment

In a fourth embodiment, an alcohol intake determining device 3 including an alcohol concentration determining unit 19 that determines whether the alcohol concentration in the breath of an occupant is equal to or higher than an allowable concentration is described.

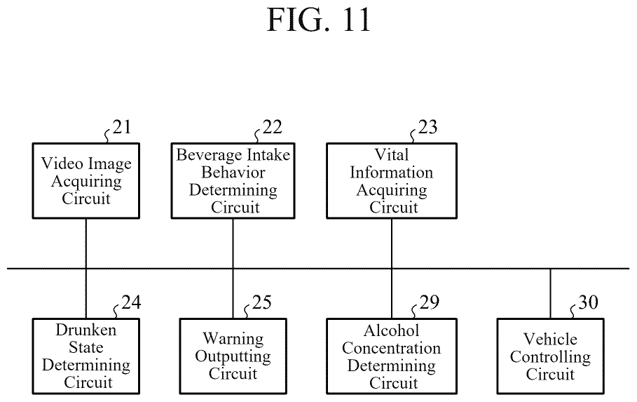

FIG. 10 is a configuration diagram illustrating the alcohol intake determining device 3 according to the fourth embodiment. In FIG. 10, the same reference numerals as those in FIG. 1, FIG. 6, and FIG. 8 denote the same or corresponding components, and therefore, detailed explanation of them is not made herein.

FIG. 11 is a hardware configuration diagram illustrating the hardware of the alcohol intake determining device 3 according to the fourth embodiment. In FIG. 11, the same reference numerals as those in FIG. 2, FIG. 7, and FIG. 9 denote the same or corresponding components, and therefore, detailed explanation of them is not made herein.

The alcohol intake determining device 3 illustrated in FIG. 10 includes a video image acquiring unit 11, a beverage intake behavior determining unit 12, a vital information acquiring unit 13, a drunken state determining unit 14, a warning outputting unit 15, the alcohol concentration determining unit 19, and a vehicle controlling unit 20.

A breath sensor 6 measures the alcohol concentration in the breath of an occupant, and outputs concentration information indicating the alcohol concentration to the alcohol intake determining device 3.

The alcohol concentration determining unit 19 is formed with an alcohol concentration determining circuit 29 illustrated in FIG. 11, for example.

The alcohol concentration determining unit 19 acquires the concentration information from the breath sensor 6.

The alcohol concentration determining unit 19 determines whether the alcohol concentration indicated by the concentration information is equal to or higher than an allowable concentration. The allowable concentration may be stored in an internal memory of the alcohol concentration determining unit 19, or may be given from the outside the alcohol intake determining device 3.

The alcohol concentration determining unit 19 outputs a determination result indicating whether the alcohol concentration is equal to or higher than the allowable concentration, to each of the video image acquiring unit 11, the vital information acquiring unit 13, and the vehicle controlling unit 20.

The vehicle controlling unit 20 is formed with a vehicle controlling circuit 30 shown in FIG. 11, for example.

The vehicle controlling unit 20 acquires, from the alcohol concentration determining unit 19, a determination result indicating whether the alcohol concentration is equal to or higher than the allowable concentration.

When the alcohol concentration determining unit 19 determines that the alcohol concentration is equal to or higher than the allowable concentration, the vehicle controlling unit 20 outputs a control signal for putting an interlock on the driving of the vehicle by the occupant, to the vehicle controlling device 4.

The alcohol intake determining device 3 illustrated in FIG. 10 is the alcohol intake determining device 3 illustrated in FIG. 1, in which the alcohol concentration determining unit 19 and the vehicle controlling unit 20 are adopted. Note that this is merely an example, and the alcohol concentration determining unit 19 and the vehicle controlling unit 20 may be adopted in the alcohol intake determining device 3 illustrated in FIG. 6 or in the alcohol intake determining device 3 illustrated in FIG. 8.

In FIG. 10, it is assumed that each of the video image acquiring unit 11, the beverage intake behavior determining unit 12, the vital information acquiring unit 13, the drunken state determining unit 14, the warning outputting unit 15, the alcohol concentration determining unit 19, and the vehicle controlling unit 20, which are components of the alcohol intake determining device 3, is formed with dedicated piece of hardware as illustrated in FIG. 11. That is, it is assumed that the alcohol intake determining device 3 is formed with the video image acquiring circuit 21, the beverage intake behavior determining circuit 22, the vital information acquiring circuit 23, the drunken state determining circuit 24, the warning outputting circuit 25, the alcohol concentration determining circuit 29, and the vehicle controlling circuit 30.

Each of the video image acquiring circuit 21, the beverage intake behavior determining circuit 22, the vital information acquiring circuit 23, the drunken state determining circuit 24, the warning outputting circuit 25, the alcohol concentration determining circuit 29, and the vehicle controlling circuit 30 is a single circuit, a composite circuit, a programmed processor, a parallel-programmed processor, an ASIC, an FPGA, or a combination thereof, for example.

The components of the alcohol intake determining device 3 are not necessarily formed with dedicated hardware, but the alcohol intake determining device 3 may be formed with software, firmware, or a combination of software and firmware.

In a case where the alcohol intake determining device 3 is formed with software, firmware, or the like, a program for causing a computer to carry out each of the processing procedures in the video image acquiring unit 11, the beverage intake behavior determining unit 12, the vital information acquiring unit 13, the drunken state determining unit 14, the warning outputting unit 15, the alcohol concentration determining unit 19, and the vehicle controlling unit 20 is stored in the memory 31 shown in FIG. 3. The processor 32 shown in FIG. 3 then executes the program stored in the memory 31.

Further, FIG. 11 illustrates an example in which each of the components of the alcohol intake determining device 3 is formed with dedicated hardware, and FIG. 3 illustrates an example in which the alcohol intake determining device 3 is formed with software, firmware, or the like. Note that this is merely an example, and some components in the alcohol intake determining device 3 may be formed with dedicated hardware while the remaining components are formed with software, firmware, or the like.

Next, an operation to be performed by the alcohol intake determining device 3 illustrated in FIG. 10 is described. Since the components other than the operations to be performed by the alcohol concentration determining unit 19 and the vehicle controlling unit 20 are the same as those of the alcohol intake determining device 3 illustrated in FIG. 1, the operations to be performed by the alcohol concentration determining unit 19 and the vehicle controlling unit 20 is mainly described herein.

The breath sensor 6 measures the alcohol concentration in the breath of the occupant at a timing when the occupant turns on the ignition switch of the vehicle after the occupant gets in the vehicle, for example. The timing at which the breath sensor 6 measures the alcohol concentration is not limited to a timing at which the occupant turns on the ignition switch, and may be a timing at which the occupant gets in the vehicle, for example.

The breath sensor 6 outputs concentration information indicating the measured alcohol concentration to the alcohol intake determining device 3.

The alcohol concentration determining unit 19 acquires the concentration information from the breath sensor 6.

The alcohol concentration determining unit 19 determines whether the alcohol concentration indicated by the concentration information is equal to or higher than the allowable concentration.

When the alcohol concentration is equal to or higher than the allowable concentration, it is necessary to stop the driver from driving the vehicle, because the driver is in a drunken state.

The alcohol concentration determining unit 19 outputs a determination result indicating whether the alcohol concentration is equal to or higher than the allowable concentration, to each of the video image acquiring unit 11, the vital information acquiring unit 13, and the vehicle controlling unit 20.

The video image acquiring unit 11 and the vital information acquiring unit 13 each acquire, from the alcohol concentration determining unit 19, a determination result indicating whether the alcohol concentration is equal to or higher than the allowable concentration.

When the determination result indicates that the alcohol concentration is equal to or higher than the allowable concentration, the driving of the vehicle by the occupant is stopped by the vehicle controlling device 4, so that the video image acquiring unit 11 does not perform a process of acquiring a captured video image.

When the determination result indicates that the alcohol concentration is equal to or higher than the allowable concentration, the driving of the vehicle by the occupant is stopped by the vehicle controlling device 4, so that the vital information acquiring unit 13 does not perform a process of acquiring vital information.

The vehicle controlling unit 20 acquires, from the alcohol concentration determining unit 19, a determination result indicating whether the alcohol concentration is equal to or higher than the allowable concentration.

When the determination result indicates that the alcohol concentration is equal to or higher than the allowable concentration, the vehicle controlling unit 20 outputs a control signal for putting an interlock on the driving of the vehicle by the occupant, to the vehicle controlling device 4.

When a control signal for putting an interlock on the driving of the vehicle is output from the vehicle controlling unit 20, the vehicle controlling device 4 does not start the engine even if the occupant performs an engine starting operation.

Further, when a control signal for putting an interlock on the driving of the vehicle is output from the vehicle controlling unit 20, the vehicle controlling device 4 causes the instrument panel of the vehicle to display a warning to the effect that driving of the vehicle is not to be performed due to the drunken state, for example.

In the fourth embodiment described above, the alcohol intake determining device 3 illustrated in FIG. 10 includes: the alcohol concentration determining unit 19 that determines whether the alcohol concentration in the breath of the occupant is equal to or higher than the allowable concentration; and the vehicle controlling unit 20 that puts an interlock on driving of the vehicle by the occupant when the alcohol concentration determining unit 19 determines that the alcohol concentration is equal to or higher than the allowable concentration. Thus, like the alcohol intake determining device 3 illustrated in FIG. 1, the alcohol intake determining device 3 illustrated in FIG. 10 can reduce erroneous detection of a drunken state more effectively than the monitoring device disclosed in Patent Literature 1, and can stop an occupant in a drunken state from driving the vehicle.

Note that, in the present disclosure, it is possible to freely combine the respective embodiments, modify any of the components of each of the embodiments, or omit any of the components in each of the embodiments.

### INDUSTRIAL APPLICABILITY

The present disclosure is suitable for an alcohol intake determining device and an alcohol intake determining method.

### REFERENCE SIGNS LIST

1: camera, 2: vital sensor, 3: alcohol intake determining device, 4: vehicle controlling device, 5: steering angle sensor, 6: breath sensor, 11: video image acquiring unit, 12: beverage intake behavior determining unit, 13: vital information acquiring unit, 14: drunken state determining unit, 15: warning outputting unit, 16: vehicle information acquiring unit, 17: beverage intake behavior determining unit, 18: drunken state determining unit, 19: alcohol concentration determining unit, 20: vehicle controlling unit, 21: video image acquiring circuit, 22: beverage intake behavior determining circuit, 23: vital information acquiring circuit, 24: drunken state determining circuit, 25: warning outputting circuit, 26: vehicle information acquisition, 27: beverage intake behavior determining circuit, 28: drunken state determining circuit, 29: alcohol concentration determining circuit, 30: vehicle controlling circuit, 31: memory, 32: processor

1. An alcohol intake determining device comprising:
a processor; and
a memory storing a program, upon executed by the processor, to perform a process:
to acquire a captured video image of an occupant in a vehicle;
to determine whether the occupant has performed an action of taking a beverage in the vehicle, on a basis of the captured video image acquired;
to acquire vital information about the occupant; and
to determine whether the occupant is under influence of alcohol, on a basis of the vital information acquired when the process determines that an action of taking a beverage has been performed.

2. The alcohol intake determining device according to claim 1, the process further comprising to output a warning to the occupant when the process determines that the occupant is under influence of alcohol.

3. The alcohol intake determining device according to claim 1, wherein
the process
detects, from the captured video image acquired, a change in position of a head of the occupant, a change in position of a hand of the occupant, and a change in angle of the head of the occupant, and determines whether the occupant has performed an action of taking a beverage in the vehicle, on a basis of the change in position of the head, the change in position of the hand, and the change in angle of the head.

4. The alcohol intake determining device according to claim 1, wherein,
when the process determines that an action of taking a beverage has been performed, the process determines whether the occupant is under influence of alcohol, on a basis of the vital information acquired or the captured video image acquired

5. The alcohol intake determining device according to claim 1, wherein
the process
acquires the vital information about the occupant from a vital sensor to measure vitality of the occupant.

6. The alcohol intake determining device according to claim 1, wherein
the process
acquires the vital information about the occupant, on a basis of the captured video image acquired.

7. The alcohol intake determining device according to claim 1, the process further comprising
to acquire vehicle information indicating a steering angle of a steering wheel of the vehicle, wherein,
only when the steering angle indicated by the vehicle information acquired is equal to or smaller than a threshold, does the process determine whether the occupant has performed an action of taking a beverage in the vehicle.

8. The alcohol intake determining device according to claim 4, wherein,
when the process has not determined that an action of taking a beverage has been performed, the process determines whether the occupant is under influence of alcohol, and determines that an abnormality has occurred in the occupant, when the occupant is determined to be under influence of alcohol.

9. The alcohol intake determining device according to claim 1, the process further comprising:
to determine whether an alcohol concentration in a breath of the occupant is equal to or higher than an allowable concentration; and
to put an interlock on driving of the vehicle by the occupant, when the process determines that the alcohol concentration is equal to or higher than the allowable concentration.

10. An alcohol intake determining method comprising:
acquiring, a captured video image of an occupant in a vehicle;
determining whether the occupant has performed an action of taking a beverage in the vehicle, on a basis of the captured video image acquired;
acquiring vital information about the occupant; and
determining whether the occupant is under influence of alcohol, on a basis of the vital information acquired, when the method determines that an action of taking a beverage has been performed.
